(19) United States
(12) Patent Application Publication
(10) Pub. No.: US 2023/0277637 A1
(43) Pub. Date: Sep. 7, 2023

(54) METHOD FOR THE TREATMENT OF A VIRAL INFECTION WITH HUMAN ALPHA-1 ANTITRYPSIN

(71) Applicant: GRIFOLS WORLDWIDE OPERATIONS LIMITED, DUBLIN (IE)

(72) Inventors: Elsa MONDOU, Dublin (IE); Susan SORRELLS, Dublin (IE); Jose Vicente TERENCIO ALEMANY, Dublin (IE); Dirk BÜSCHER, Dublin (IE); Maria Del Carmen CARMONA OROZCO, Dublin (IE); Mireia TORRES HURTADO, Dublin (IE)

(21) Appl. No.: 17/999,362

(22) PCT Filed: May 26, 2021
(86) PCT No.: PCT/EP2021/064015

§ 371 Date: Nov. 18, 2022

Related U.S. Application Data
(60) Provisional application No. 63/030,548, filed on May 27, 2020.

(30) Foreign Application Priority Data
May 27, 2020 (EP) 20382452.9

(51) Int. Cl. A61K 38/57

(52) U.S. Cl. CPC A61K 38/57

(57) ABSTRACT

A method is for the treatment of a viral infection with human alpha-1 antitrypsin (A1AT). Methods and compositions for the treatment of coronavirus disease 2019 (COVID-19) in a patient, include administering to the patient a therapeutically effective amount of human alpha-1 antitrypsin. The A1AT is administered in an amount between 60 mg/kg and 200 mg/kg. Methods and compositions for the treatment of Cytokine Release Syndrome (CRS) in a patient, include administering to the patient a therapeutically effective amount of an inhibitor of interleukin-6 in combination with a therapeutically effective amount of A1AT.

### DESCRIPTION

The present disclosure is related to the field of pharmaceutical products. In particular, the present application refers to methods and compositions for the treatment of Coronavirus Disease 19 (COVID-19) in a patient in need thereof, the method comprising administering to the patient a therapeutically effective amount of human α-1-proteinase inhibitor (A1AT). The present invention also relates to methods and compositions for the treatment of Cytokine Release Syndrome (CRS) in a patient in need thereof, the method comprising administering to the patient a therapeutically effective amount of an inhibitor of interleukin-6 (IL-6), such as tocilizumab, and human α-1-proteinase inhibitor (A1AT).

Human αproteinase inhibitor (A1AT) is a plasma protein fraction also known as human αantitrypsin or αproteinase. A1AT is the most abundant multifunctional serine proteinase inhibitor in human plasma. It is an acute-phase glycoprotein, and its best characterized target is neutrophil elastase (although other serine proteases like proteinase 3 and cathepsin G have been reported as potential targets).

A1AT is indicated for chronic augmentation and maintenance therapy in individuals with alpha1-proteinase inhibitor deficiency and clinical evidence of emphysema. This is a rare genetic disease often called “genetic COPD”, where patients do not generate enough A1AT. This deficiency is associated with lung and liver diseases (e.g. reduced lung function, emphysema, infections...), probably due to the proteinase-antiproteinase imbalance that results in tissue damage caused by the excessive proteinase activity.

In addition to the antiproteinase dependent tissue damage protection, scientific studies published in the last years suggest that A1AT may also play a role as an antiinflammatory and an immunoregulatory protein. Indeed, preliminary findings in animals have suggested that A1AT could be used for the management of diseases not necessarily related to inherited A1AT deficiency and several clinical trials have been carried out to test the use of A1AT in other indications. Some examples of indications are:
Diabetes Mellitus type 1
HIV infection
Neuromyelitis optica
Acute Myocardial infarction (AMI)
Graft vs Host disease (transplantation)
Pancreatitis
Lung transplantation rejection
Cystic fibrosis
COPD
Post Cardiac Surgery Systemic Inflammatory Response

On the other hand, coronaviruses are a large family of positive-sense single-stranded RNA viruses which may cause illness in animals or humans. In humans, several coronaviruses are known to cause respiratory infections ranging from the common cold to more severe diseases such as Middle East Respiratory Syndrome (MERS) and Severe Acute Respiratory Syndrome (SARS). The most recently discovered coronavirus, SARS-CoV-2, causes the associated coronavirus disease COVID-19. This new virus and disease were unknown before the outbreak began in Wuhan, China, in December 2019.

The most common symptoms of COVID-19 are fever, tiredness, and dry cough. Some patients may have aches and pains, nasal congestion, runny nose, sore throat, or diarrhea. These symptoms are usually mild and begin gradually. Some people become infected but do not develop any symptoms and do not feel unwell. The disease can spread through respiratory droplets produced when an infected person coughs or sneezes. These droplets land on objects and surfaces around the person. Other people may acquire SARS-CoV-2 by touching these objects or surfaces, then touching their eyes, nose, or mouth.

Person to person spread was subsequently reported worldwide. The World Health Organization (WHO) has designated the pandemic of COVID-19 a Public Health Emergency of International Concern.

Currently there are no approved treatments for COVID-19 in Europe or the United States. The lack of disease-directed therapeutic options has led to urgent interventions in anticipation of some potentially promising effects. Some antivirals are currently under evaluation. These include favipirivir (AVIGAN) manufactured by Fujifilm in Japan, remdesivir manufactured by Gilead, and Kaletra® (lopinavir/ritonavir) commercially available for human immunodeficiency virus (HIV). There are also investigations of chloroquine and hydroxychloroquine as treatment modalities and potential applications for post-exposure prophylaxis according to Clinicaltrials.gov and other clinical trial registries. These and other potential therapeutic agents are described on the World Health Organization (WHO) website file: WHO Landscape Therapeutics under investigation 17 Feb. 2020.pdf (accessed 19 Mar. 2020).

On the other hand, tocilizumab is a biologic medication currently approved to treat adults with moderately to severely active rheumatoid arthritis (RA), adults with giant cell arteritis (GCA), and children ages two years old and above with Polyarticular Juvenile Idiopathic Arthritis (PJIA) or Systemic Juvenile Idiopathic Arthritis (SJIA). Biologic medications are proteins designed by humans that affect the immune system. Tocilizumab blocks the inflammatory protein interleukin-6 (IL-6). This improves joint pain and swelling from arthritis and other symptoms caused by inflammation.

Cytokine release syndrome (CRS) is a systemic inflammatory response syndrome (SIRS) that can be triggered by a variety of factors such as infections, biological drugs, and transplantations. CRS occurs when large numbers of immune cells are activated to release inflammatory cytokines, which in turn activate more immune cells in an expanding cycle of inflammation.

When cytokines are released into the circulation, systemic symptoms such as fever, nausea, chills, hypotension, tachycardia, asthenia, headache, rash, scratchy throat, and dyspnea can result. In most patients, the symptoms are mild to moderate in severity and are managed easily. Patients with severe CRS are treated with drugs that counteract the immune response. These medicines include targeted therapies to block specific cytokines, as well as more general immunosuppressive drugs.

Accordingly, there is a need for methods and compositions for effectively treating COVID-19 in patients in need thereof, that can reduce the proportion of subjects dying or requiring intensive care unit (ICU) admission on or before Day 29 or who are dependent on high flow oxygen devices or invasive mechanical ventilation on Day 29 versus SMT alone in hospitalized subjects with COVID-19.

The present inventors have also surprisingly found a synergistic effect of a composition comprising tocilizumab and A1AT for the treatment of CRS in patients in need thereof, that is, a combined therapy using these two pharmaceutical components will improve the treatment of said condition. After a simulation of the predicted effect on CRS of A1AT in combination with about 3000 approved medicaments, the higher effect (95 %) was combination with tocilizumab.

In a first aspect, the present disclosure provides methods and compositions for the treatment of COVID-19 in a patient in need thereof. In some embodiments, the method comprises administering to the patient a therapeutically effective amount of human alpha-1 antitrypsin (A1AT) wherein A1AT is administered in an amount between 60 mg/kg and 200 mg/kg.

In some embodiments, the patient is also subjected to standard medical treatment (SMT) for COVID-19.

In some embodiments, the COVID-19 can be mild, moderate or severe.

In some embodiments, the composition comprising A1AT is administered intravenously.

In some embodiments, A1AT can be administered in an amount between 80 mg/kg and 160 mg/kg, most preferably 120 mg/kg.

The composition of the present invention can be administered at least every 6 hours, or at least every 8 hours, or at least every 12 hours, or at least every 24 h, or at least every 48 hours, or at least every 72 hours, or at least once a week, or at least once every two weeks. It is also contemplated that the composition of the present invention is administered as a single dose.

In some embodiments, the patient in need thereof is a hospitalized male or female subject ≥ 18 years of age at time of screening who is being treated for COVID-19. In some embodiments, subjects are screened within 48 hours (≤ 48 hours) of hospital admission.

In some embodiments, the patient in need thereof has laboratory-confirmed novel coronavirus (SARS-CoV-2) infection as determined by as determined by any nucleic acid technology (NAT) or any other commercial or public health assay.

In some embodiments of the method of the present invention the patient is positive for SARS-CoV-2 infection as determined by any nucleic acid technology (NAT) or any other commercial or public health assay. In some preferred embodiments, the nucleic acid technology is any amplification or transcription-based technique known in the art. In more preferred embodiments, said nucleic acid technology is selected from the group including PCR, RT-PCR, strand displacement amplification (SDA), thermophilic SDA (tSDA), rolling circle amplification (RCA), helicase dependent amplification (HDA), loop-mediated isothermal amplification (LAMP), nucleic acid sequence based amplification (NASBA), Qβ replicase, self-sustained sequence replication and transcription-mediated amplification (TMA). In more preferred embodiments, the nucleic acid technology is PCR, RT-PCR or TMA.

In some embodiments, the patient in need thereof has COVID-19 illness (symptoms) of any duration.

In another aspect, the present disclosure also provides methods and compositions for the treatment of Cytokine Release Syndrome (CRS) in a patient in need thereof. In some embodiments, the method comprises administering to the patient a therapeutically effective amount of an inhibitor of interleukin-6 (IL-6) in combination with a therapeutically effective amount of A1AT.

In some embodiments, the IL-6 inhibitor is tocilizumab.

In some embodiments, the CRS can be mild, moderate or severe.

In some embodiments, the CRS is caused by a viral infection, or by a bacterial infection, or by a fungal infection, or by a protozoal infection, or by a combination thereof.

In some embodiments, the CRS is caused by the SARS-COV-2 virus.

In some embodiments, the composition is administered intravenously.

In some embodiments, tocilizumab can be administered in an amount of about 4 mg/kg to about 8 mg/kg, preferably in an amount of about 5 mg/kg to about 7 mg/kg, at a dose of 400 mg/dose to 800 mg/dose, preferable between 500 mg/dose and 700 mg/dose.

In some embodiments, A1AT can be administered in an amount between 60 mg/kg and 200 mg/kg, preferably between 80 mg/kg and 160 mg/kg, most preferably 120 mg/kg.

The composition of the present invention can be administered at least every 6 hours, or at least every 8 hours, or at least every 12 hours, or at least every 24 hours, or at least every 48 hours, or at least every 72 hours, or at least once a week, or at least once every two weeks. Preferably, the composition is administered twice, one week after the first administration. It is also contemplated that the composition of the present invention is administered as a single dose.

In another aspect, the present invention refers to method and composition for the treatment of Acute Respiratory Distress Syndrome (ARDS) resulting from CRS, the method comprises administering to the patient a therapeutically effective amount of an inhibitor of interleukin-6 (IL-6) in combination with a therapeutically effective amount of A1AT.

In some embodiments, the IL-6 inhibitor is tocilizumab.

In some embodiments, the composition is administered intravenously.

In some embodiments, tocilizumab can be administered in an amount of about 4 mg/kg to about 8 mg/kg, preferably in an amount of about 5 mg/kg to about 7 mg/kg, at a dose of 400 mg/dose to 800 mg/dose, preferable between 500 mg/dose and 700 mg/dose.

In some embodiments, A1AT can be administered in an amount between 60 mg/kg and 200 mg/kg, preferably between 80 mg/kg and 160 mg/kg, most preferably 120 mg/kg.

The composition of the present invention can be administered at least every 6 hours, or at least every 8 hours, or at least every 12 hours, or at least every 24 hours, or at least every 48 hours, or at least every 72 hours, or at least once a week, or at least once every two weeks. Preferably, the composition is administered twice, one week after the first administration. It is also contemplated that the composition of the present invention is administered as a single dose.

In a further aspect, the present invention refers to method and composition for the treatment of Septic Shock, the method comprises administering to the patient a therapeutically effective amount of an inhibitor of interleukin-6 (IL-6) in combination with a therapeutically effective amount of A1AT.

In some embodiments, the IL-6 inhibitor is tocilizumab.

In some embodiments, the composition is administered intravenously.

In some embodiments, tocilizumab can be administered in an amount of about 4 mg/kg to about 8 mg/kg, preferably in an amount of about 5 mg/kg to about 7 mg/kg, at a dose of 400 mg/dose to 800 mg/dose, preferable between 500 mg/dose and 700 mg/dose.

In some embodiments, A1AT can be administered in an amount between 60 mg/kg and 200 mg/kg, preferably between 80 mg/kg and 160 mg/kg, most preferably 120 mg/kg.

The composition of the present invention can be administered at least every 6 hours, or at least every 8 hours, or at least every 12 hours, or at least every 24 hours, or at least every 48 hours, or at least every 72 hours, or at least once a week, or at least once every two weeks. Preferably, the composition is administered twice, one week after the first administration. It is also contemplated that the composition of the present invention is administered as a single dose.

### Definitions

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

As used in this specification and claims, the singular forms “a,” “an” and “the” include plural references unless the content clearly dictates otherwise.

As used herein, “about” means a quantity, level, value, number, frequency, percentage, dimension, size, amount, weight or length that varies by as much as 20, 15, 10, 9, 8, 7, 6, 5, 4, 3, 2 or 1 % to a reference quantity, level, value, number, frequency, percentage, dimension, size, amount, weight or length.

The term “treatment” or “treating” means any treatment of a disease or disorder in a subject, such as a mammal, including: preventing or protecting against the disease or disorder, that is, causing the clinical symptoms not to develop; inhibiting the disease or disorder, that is, arresting or suppressing the development of clinical symptoms; and/or relieving the disease or disorder that is, causing the regression of clinical symptoms.

It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein.

The term “therapeutically effective amount” refers to that amount of A1AT, typically delivered as pharmaceutical compositions, that is sufficient to effect treatment, as defined herein, when administered to a subject in need of such treatment.

In the present invention, the term “standard medical treatment” (SMT) refers to a treatment that is accepted by medical experts as a proper treatment for a certain type of disease, and that is widely used by healthcare professionals. In the context of the present invention, SMT refers to the standard treatment for COVID-19 patients that is been used in the medical centre where the treatment with A1AT the present invention is used. Thus, STM may include treatments with some of the potential therapeutic agents described on the World Health Organization (WHO) but it may also include other treatments not accepted by the WHO.

The term “Nucleic acid technology or NAT”, as used herein, refers to any amplification-based or transcription-based method for detection and quantitation of a target nucleic acid. Numerous amplification-based methods are well known and established in the art, such as PCR, its variation RT-PCR, strand displacement amplification (SDA), thermophilic SDA (tSDA), rolling circle amplification (RCA), helicase dependent amplification (HDA), or loop-mediated isothermal amplification (LAMP). Transcription-based amplification methods commonly used in the art include nucleic acid sequence based amplification (NASBA), Qβ replicase, self-sustained sequence replication or transcription-mediated amplification (TMA).

Although this disclosure is in the context of certain embodiments and examples, those skilled in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of skill in the art based upon this disclosure.

It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above.

It should be understood, however, that this description, while indicating preferred embodiments of the disclosure, is given by way of illustration only, since various changes and modifications within the spirit and scope of the disclosure will become apparent to those skilled in the art.

The terminology used in the description presented herein is not intended to be interpreted in any limited or restrictive manner. Rather, the terminology is simply being utilized in conjunction with a detailed description of embodiments of the systems, methods and related components. Furthermore, embodiments may comprise several novel features, no single one of which is solely responsible for its desirable attributes or is believed to be essential to practicing the embodiments herein described.

### AlphaAntitrypsin

AlphaProteinase inhibitor (A1PI), also known as AlphaAntitrypsin (AAT) or α1-Antitrypsin, is a proteinase inhibitor that acts on a variety of cellular proteases. A1PI plays a major role in tissue homeostasis through the inhibition of neutrophil elastase action, and through other mechanisms.

Congenital deficiencies of A1PI allow uncontrolled activity of neutrophil elastase and the subsequent degradation of elastin, an essential protein that confers elasticity to tissues, particularly the lungs. The absence of elastin may result in respiratory complications such as pulmonary emphysema and hepatic cirrhosis. Chronic intravenous (IV) administration of A1PI is usually used for the treatment of AAT deficiency.

A1PI can be obtained from human plasma, using the Cohn Fractionation Process, as it is known to the skilled person. However, the A1PI can be obtained from other sources and using different techniques, such as recombinant DNA technique. One suitable example of a pharmaceutical product of A1PI is commercialized under the trade name Prolastin-C (Grifols S.A., Spain).

### Tocilizumab

Tocilizumab (TCZ), is a recombinant humanized, anti-human monoclonal antibody of the immunoglobulin G1k subclass directed against soluble and membrane-bound interleukin 6 receptors (IL 6R). It is marketed in the European Union (EU) under the trade name RoActemra and in the United States as Actemra (Hoffmann-La Roche, Switzerland). TCZ was first approved in 2005 as an orphan drug in Japan for the treatment of Castleman’s disease, a rare lymphoproliferative disease involving expansion of plasma cell numbers. TCZ is now licensed in the EU for use alone or in combination with disease-modifying anti-rheumatic drugs (DMARDs) to treat adult patients with moderate to severely active rheumatoid arthritis (RA), children over 2 y of age with the systemic form of juvenile idiopathic arthritis (sJIA) or children over 2 y of age with the polyarticular form of Juvenile Idiopathic Arthritis (pJIA). It has also been studied as a treatment of other conditions such as Crohn’s disease, systemic lupus erythematosus, Takayasu Arteritis (TA), Giant Cell Arteritis (GCA) Polymyalgia Rheumatica (PMR) and refractory adult-onset still disease.

### EXAMPLE

### Example 1. Simulation of the Predicted Effect of the Method of the Present Invention on CRS

To identify new indications of A1AT in combination with other drugs a prediction technology was used. This technology, which is known to the skilled person, creates mathematical models that integrate biochemical, pharmaceutical, and clinical data with the aim of simulating human physiology. The analysis of these models provides mechanistic insight into biological processes and has the potential to uncover non-obvious patterns and molecular explanations.

In this case, a repositioning screening was performed, where the efficacy of combinations of A1AT with other approved drugs (aprox 3000 drugs; source Drugbank database) were computationally predicted by calculating their molecular impact on a set of mathematical models simulating different indications.

A1AT was molecularly characterized: the main proteins, including known A1AT targets and off-targets mediating the known downstream effect of the A1AT.

Once the mathematical models were generated, a drug repositioning analysis was performed on them in order to calculate the efficacy of each combination of A1AT with an existing approved drug for treating each disease. Approximately, 3000 drug combinations were tested on each indication. They were ranked according to the degree of predicted modulation of the indication (100 - 0 %). From all the 3000 combinations made, the higher effect (95 %) on the treatment of CRS was obtained with A1AT in combination with tocilizumab, which is higher than the predicted effect for each of these drugs separately, i.e. 67 % and 47 %, respectively.

The following table shows the predicted effect on Cytokyne Release Syndrome of each drug separately and in combination.

| Drug A | Drug B | Predicted effect of Drug A | Predicted effect of Drug B | Predicted effect of Drug A + Drug B |
|---|---|---|---|---|
| Tocilizumab | A1AT | 47 % | 67 % | 95 % |
| Esmolol | A1AT | 76 % | 67 % | 78 % |
| Lutropin α | A1AT | 78 % | 67 % | 79 % |
| Cimetidine | A1AT | 74 % | 67 % | 79 % |

What is claimed is:

1. A method for the treatment of coronavirus disease 2019 (COVID-19) in a patient in need thereof, comprising administering to the patient a therapeutically effective amount of human alpha-1 antitrypsin (A1AT), wherein A1AT is administered in an amount between 60 mg/kg and 200 mg/kg.

2. The method according to claim 1, wherein the patient is also subjected to standard medical treatment (SMT) for COVID-19.

3. The method according to claim 1, wherein the COVID-19 is mild, moderate or severe.

4. The method according to claim 1, wherein the composition comprising A1AT is administered intravenously.

5. The method according to claim 1, wherein the composition comprising A1AT is administered in an amount between 80 mg/kg and 160 mg/kg.

6. The method according to claim 5, wherein the composition comprising A1AT is administered in an amount of 120 mg/kg.

7. The method according to claim 1, wherein the composition comprising A1AT is administrated at least every 6 hours, or at least every 8 hours, or at least every 12 hours, or at least every 24h, or at least every 48 hours, or at least every 72 hours, or at least once a week, or at least once every two weeks.

8. The method according to claim 1, wherein the composition comprising A1AT is administered as a single dose.

9. The method according to claim 1, wherein the patient in need thereof is a hospitalized male or female subject ≥ 18 years of age at time of screening who is being treated for COVID-19.

10. The method according to claim 1, wherein the patient in need thereof has laboratory-confirmed novel coronavirus (SARS-CoV-2) infection as determined by any nucleic acid technology (NAT) or any other commercial or public health assay.

11. The method according to claim 1, wherein the patient in need thereof has COVID-19 illness (symptoms) of any duration.

12-54. (canceled)

55. A method for the treatment of Acute Respiratory Distress Syndrome (ARDS) resulting from CRS, the method comprises administering to the patient a composition according to claim 57.

56. A method for the treatment of Septic Shock, the method comprises administering to the patient a compostion according to claim 58.

57. A composition comprising a therapeutically effective amount of an inhibitor of interleukin-6 (IL-6) in combination with a therapeutically effective amount of human alpha-1 antitrypsin (A1AT) for use in the treatment of Acute respiratory Distress Syndrome (ARDS).

58. A composition comprising a therapeutically effective amount of an inhibitor of interleukin-6 (IL-6) in combination with a therapeutically effective amount of human alpha-1 antitrypsin (A1AT) for use in the treatment of Septic Shock.

59. The method according to claim 55, wherein said composition is administered intravenously.

60. The method according to claim 55, wherein the inhibitor of interleukin-6 (IL-6) is administered to the patient in an amount of about 4 mg/kg to about 8 mg/kg.

61. The composition according to claim 57, wherein A1AT is administered to a subject in an amount between 60 mg/kg and 200 mg/kg.

62. The composition according to claim 57, wherein a subject the A1AT is administered is also subjected to a standard medical treatment (SMT) for COVID-19.

63. The composition according to claim 57, wherein the composition is administered intravenously.
